(19) United States
(12) Patent Application Publication
(10) Pub. No.: US 2005/0287101 A1
(43) Pub. Date: Dec. 29, 2005

(54) Cosmetic composition comprising at least one semi-crystalline polymer, and at least one ester of dimer diol and of acid

(76) Inventors: Caroline Lebre, Thiais (FR); Véronique Ferrari, Maisons-Alfort (FR)

Correspondence Address: FINNEGAN, HENDERSON, FARABOW, GARRETT & DUNNER;LLP, 901 NEW YORK AVENUE, NW, WASHINGTON, DC, 20001-4413, US

(21) Appl. No.: 11/147,353

(22) Filed: Jun. 8, 2005

Related U.S. Application Data
(60) Provisional application No. 60/580,365, filed on Jun. 18, 2004.

(30) Foreign Application Priority Data
Jun. 8, 2004 (FR) 04 06168

(51) Int. Cl. A61K 7/06; A61K 7/11

(52) U.S. Cl. 424/70.16

(57) ABSTRACT

The present disclosure relates to a cosmetic composition comprising at least one ester of dimer diol and of at least one acid chosen from dicarboxylic and C₄ to C₃₄ monocarboxylic acids, and at least one semi-crystalline polymer with an organic structure, wherein the at least one semi-crystalline polymer has a melting point greater than or equal to 30° C.

This application claims benefit of U.S. Provisional Application No. 60/580,365, filed Jun. 18, 2004, the contents of which are incorporated herein by reference. This application also claims benefit of priority under 35 U.S.C. § 119 to French Patent Application No. 04 06168, filed Jun. 8, 2005, the contents of which are also incorporated herein by reference.

The present disclosure relates to a cosmetic composition, for instance a cosmetic composition for making up or caring for the skin, both of the face and of the human body, including the scalp, lips or superficial body growths of human beings, such as the hair, eyelashes, eyebrows or nails, comprising a cosmetically acceptable medium.

The composition of the present disclosure can, for example, constitute a product for making up the body, lips or superficial body growths of human beings, having non-therapeutic treatment and/or care properties. For example, the composition can constitute a lipstick or a lip gloss, a face powder or eye shadow, a tattooing product, a mascara, an eyeliner, a nail varnish, a product for the artificial tanning of the skin, or a product for coloring or caring for the hair.

There exist numerous cosmetic compositions for which gloss properties are desirable for the film deposited, after application to keratinous substances (skin, lips, superficial body growths). Mention may be made, for example, of lipsticks, nail varnishes or certain hair products. To bring about the desired gloss, it is known in the art to use, as an active agent in terms of gloss, lanolins in combination with at least one “glossy” oil, such as oily polymers; for example polybutenes, which have a high viscosity; fatty alcohol or acid esters, the carbon number of which is high (typically greater than 16); or certain vegetable oils.

However, the glossy compositions of the prior art can exhibit the disadvantage of having insufficient hold over time, for example they may migrate out of the initial outline of the makeup.

There is therefore a need for cosmetic makeup or care compositions forming a deposited layer which exhibits good hold on contact with liquids brought into contact with the makeup, such as during a meal, and which does not migrate.

Accordingly, the present disclosure relates to compositions comprising at least one ester of dimer diol and of mono- or dicarboxylic acids, which beneficially make it possible to provide satisfactory gloss while improving the nonmigration of the cosmetic composition.

Esters of dimer diols and of mono- or dicarboxylic acids have been disclosed, for example, in French Patent No. FR 2 795 309, as being useful in the preparation of cosmetic compositions having, for example, improved stability properties. More recently, Japanese Patent Nos. JP 2002-128623, 2002-128628, and 2002-128629 provide for cosmetic compositions, such as makeup compositions, which include, as active agent for the gloss, esters of dilinoleic diacids with dilinoleic dimer diols.

The inventors have discovered, surprisingly, that compositions comprising the combination of at least one ester of dimer diol and of acid, and at least one specific semi-crystalline polymer, can be glossy and have good hold. For example, such compositions may not migrate.

Consequently, the present disclosure relates to a cosmetic composition comprising, in a physiologically acceptable medium, at least one ester of dimer diol and of at least one acid chosen from dicarboxylic and C₄ to C₃₄ monocarboxylic acids, and at least one semi-crystalline polymer, wherein the at least one semi-crystalline polymer has a melting point greater than or equal to 30° C.

The present disclosure also relates to a method for making up and/or caring for the keratin materials comprising applying to the keratin materials at least one composition according to the present disclosure, wherein the keratin materials are chosen from the skin, lips and/or superficial body growths.

Esters of Dimer Diol and of Acid

The esters of dimer diol and of acid that can be used in the context of the present disclosure are available commercially, and can be prepared conventionally. They can be, for example, of vegetable origin and can be obtained by esterification of a dimer diol with a C₄-C₃₄ monocarboxylic acid, such as, for example, a fatty acid, or with a dicarboxylic acid, such as a dimer diacid.

The esters of dimer diol and of acid obtained by esterification with a monocarboxylic acid can have a relatively high molecular weight, ranging, for example, from 1,000 g/mol to 1,300 g/mol. A dimer diol dicarboxylate can be obtained which exhibits a weight-average molecular weight, determined by gel permeation chromatography (GPC), ranging from 2,000 g/mol to 20,000 g/mol, such as ranging from 2,000 g/mol to 4,000 g/mol.

The monocarboxylic acids that can be used according to the present disclosure comprise from 4 to 34 carbon atoms, for instance from 10 to 32 carbon atoms.

Among the examples of monocarboxylic acids that may be used as disclosed herein, non-limiting mention may be made of:
saturated linear acids, such as butanoic acid, pentanoic acid, hexanoic acid, heptanoic acid, octanoic acid, nonanoic acid, decanoic acid, undecanoic acid, dodecanoic acid, tridecanoic acid, tetradecanoic acid, heptadecanoic acid, hexadecanoic acid, pentadecanoic acid, octadecanoic acid, nonadecanoic acid, eicosanoic acid, docosanoic acid or tetracosanoic acid,
branched fatty acids, such as, for example, isobutanoic acid, isopentanoic acid, pivalic acid, isohexanoic acid, isoheptanoic acid, isooctanoic acid, dimethyloctanoic acid, isononanoic acid, isodecanoic acid, isoundecanoic acid, isododecanoic acid, isotridecanoic acid, isotetradecanoic acid, isopentadecanoic acid, isohexadecanoic acid, isoheptadecanoic acid, isooctadecanoic acid, isononadecanoic acid, isoeicosanoic acid, 2-ethylhexanoic acid, 2-butyloctanoic acid, 2-hexyldecanoic acid, 2-octyldodecanoic acid, 2-decyltetradecanoic acid, 2-dodecylhexadecanoic acid, 2-tetradecyloctadecanoic acid, 2-hexadecyloctadecanoic acid or long-chain fatty acids obtained from lanolin,
unsaturated linear C₈ to C₃₄ fatty acids, such as undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid or arachidonic acid,
hydroxy acids, such as 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytridecanoic acid, 2-hydroxytetradecanoic acid, 2-hydroxyhexadecanoic acid, 2-hydroxyheptadecanoic acid, 2-hydroxyoctadecanoic acid, 12-hydroxyoctadecanoic acid, 2-hydroxynonadecanoic acid, 2-hydroxyeicosanoic acid, 2-hydroxydocosanoic acid or 2-hydroxytetracosanoic acid,
cyclic acids, such as cyclohexanoic acid, hydrogenated rosin, rosin, abietic acid, hydrogenated abietic acid, benzoic acid, p-hydroxybenzoic acid, p-aminobenzoic acid, cinnamic acid, p-methoxycinnamic acid, salicylic acid, gallic acid, pyrrolidonecarboxylic acid or nicotinic acid, and
fatty acids of natural origin, such as fatty acids of orange oil, of avocado oil, of macadamia oil, of olive oil, of hydrogenated soybean oil, of jojoba oil, of palm oil, of castor oil, of wheat germ oil, of saffron oil, of cottonseed oil or of mink oil, and their mixtures.

For example, in one embodiment of the present disclosure, the monocarboxylic acids are chosen from fatty acids, such as those defined above. As used herein, the term “fatty acid” is understood to mean a carboxylic acid obtained by hydrolysis of vegetable or animal fats or oils. The fatty acid can be saturated or unsaturated.

The at least one ester obtained can be chosen from diesters, and monoesters. For instance, the at least one ester can be a mixture of at least two ester types formed with different carboxylic acids.

The dicarboxylic acids that can be used according to the present disclosure comprise at least two carboxyl groups per molecule. For example, the dicarboxylic acids can be chosen from those of formula (I):
HOOC—(CH₂)ₙ—COOH (I)
wherein n is an integer ranging from 1 to 16, such as from 3 to 16.

Among the dicarboxylic acids that may be used as disclosed herein, non-limiting mention may be made of: malonic acid, succinic acid, glutaric acid, adipic acid, pimelic acid, suberic acid, azalaic acid, sebacic acid, 1,9-nonamethylenedicarboxylic acid, 1,10-decamethylenedicarboxylic acid, 1,11-undecamethylenedicarboxylic acid, 1,12-dodecamethylenedicarboxylic acid, 1,13-tridecamethylenedicarboxylic acid, 1,14-tetradecamethylenedicarboxylic acid, 1,15-pentadecamethylenedicarboxylic acid, 1,16-hexadecamethylenedicarboxylic acid, and mixtures thereof.

The dicarboxylic acid can also be chosen from dimer diacids. As used herein, the term “dimer diacid” is understood to mean a diacid obtained by an intermolecular polymerization, for instance dimerization, reaction of at least one unsaturated monocarboxylic acid.

They can derive, for example, from the dimerization of an unsaturated fatty acid, for instance unsaturated C₈ to C₃₄ fatty acids, such as unsaturated C₁₂ to C₂₂ fatty acids, and unsaturated C₁₆ to C₂₀ fatty acids, such as an unsaturated C₁₈ fatty acid.

Mention may be made, by way of non-limiting example, of the unsaturated fatty acids, as above, including undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid, arachidonic acid, and mixtures thereof.

According to one embodiment of the present disclosure, for example, the dimer diacid is that from which the dimer diol to be esterified also derives. For instance, the dimer diacid can be obtained by dimerization of linoleic acid, optionally followed by hydrogenation of the carbon-carbon double bonds. The dimer diacid can be in the saturated form, that is to say it does not need to comprise any carbon-carbon double bond.

According to another embodiment of the present disclosure, the possible carbon-carbon double bonds of the dimer diacid are all or partly hydrogenated, after the esterification reaction of the dimer diacid with the dimer diol.

According to yet another embodiment of the present disclosure, the dimer diacid is a commercial product comprising a dicarboxylic acid comprising 36 carbon atoms. This product also comprises a trimeric acid and a monomeric acid, in amounts that depend on the degree of purity of the product. Conventionally, products with dimer diacid present in an amount greater than or equal to 70%, and others wherein dimer diacid is present in an amount adjusted to greater than or equal to 90% are available commercially.

Dimer diacids, for instance dilinoleic diacids, the stability of which with regard to oxidation has been improved by hydrogenation of the double bonds remaining after the dimerization reaction, are also available commercially.

Any dimer diacid currently available commercially can be used according to the present disclosure.

In an esterification reaction with a dicarboxylic acid, such as a dimer diacid, the mean degree of esterification and the average molecular weight of the ester obtained can be adjusted by varying the ratio of the dimer diol to the dicarboxylic acid, for example to the dimer diacid. The ratio, expressed as the molar proportion of the dicarboxylic acid, based on the average molecular weight calculated from its acid number, per 1 mol of dimer diol, based on the average molecular weight calculated from its hydroxyl number, can range from 0.2 mol to 1.2 mol, such as from 0.4 mol to 1.0 mol, for example equal to 0.5 mol or 0.7 mol.

As used herein, the term “dimer diol” is understood to mean, for example, saturated diols produced by hydrogenation of the corresponding dimer diacids, a dimer diacid being as defined above.

With respect to dimer diols manufactured industrially, they may also comprise other components, for example a trimer triol, a monoalcohol and compounds of ether type, depending on the degree of purification of the dimeric acid and/or of the lower alcohol ester of the latter used as starting material. Generally, products with dimer diol present in an amount greater than or equal to 70% can be used in the present disclosure. However, for example, dimer diol of high purity, such as a compound with dimer diol present in an amount greater than or equal to 90%, may also be used.

Thus, a dimer diol can be produced by catalytic hydrogenation of a dimer diacid, itself obtained by dimerization of at least one unsaturated fatty acid, for example, an unsaturated C₈ to C₃₄ fatty acid, such as those mentioned above, for instance an unsaturated C₁₂ to C₂₂ fatty acid, such as an unsaturated C₁₆ to C₂₀ fatty acid, and for instance an unsaturated C₁₈ fatty acid, such as, for example, oleic acid and linoleic acid.

According to one embodiment of the present disclosure, the dimer diol derives from the hydrogenation of the acid functional groups of dilinoleic diacid.

For example, it can be the dimer diol obtained by dimerization of linoleic acid, followed by hydrogenation of the acid functional groups. The dimer diol can be in the saturated form, that is to say may not comprise any carbon-carbon double bonds. According to another embodiment of the present disclosure, the possible carbon-carbon double bonds of the dimer diol are all or partly hydrogenated, after the esterification reaction of the dimer diacid with the dimer diol.

According to still another embodiment of the present disclosure, the dimer diol ester is an ester of dimer diol and of dimer diacid, for example, chosen from those of formula (II):
HO—R¹—(—OCO—R²—COO—R¹-)ₕ—OH (11)
wherein:
R¹ is a dimer diol residue obtained by hydrogenation of dilinoleic diacid,
R² is a hydrogenated dilinoleic diacid residue, and
h is an integer ranging from 1 to 9.

Non-limiting mention may be made, by way of illustration of the esters suitable for use in the present disclosure, of the esters of dilinoleic diacids and of dilinoleic dimer diols sold by Nippon Fine Chemical under the trade names Lusplan DD-DA5® and DD-DA7®.

The amount of ester according to the present disclosure can be adjusted so as to control the mean gloss of the composition at the desired value. For example, the ester can be present in an amount ranging from 1% to 99%, for instance ranging from 2% to 60% by weight, such as from 5% to 40%, and from 10% to 35% by weight, relative to the total weight of the composition.

The compositions of the present disclosure can be provided in a form chosen from paste, solid or cream form. It can also be an oil-in-water or water-in-oil emulsion, or a solid or soft anhydrous gel. For example, it may be provided in an anhyrous form, such as in the form of an anhydrous gel, for instance cast as a stick or in a dish.

Semi-crystalline Polymer

As used herein, the term “polymers” is understood to mean compounds comprising at least 2 repeat units, for instance at least 3 repeat units, such as at least 10 repeat units.

As used herein the term “semi-crystalline polymer” is understood to mean polymers comprising at least one crystallizable part and at least one amorphous part in the backbone and exhibiting a first-order reversible phase change temperature, such as a melting point (solid-liquid transition). The at least one crystallizable part is either a side chain (or a pendent chain) or a block in the backbone.

When the at least one crystallizable part of the semi-crystalline polymer is a block of the polymer backbone, this crystallizable block is different in chemical nature from the amorphous blocks; in this case, the semi-crystalline polymer is a block copolymer, for example of the diblock, triblock or multiblock type. When the at least one crystallizable part is a chain pendent to the backbone, the semi-crystalline polymer can be a homopolymer or a copolymer.

As used herein, the terms “organic compound” or “with an organic structure” is understood to mean compounds comprising carbon atoms and hydrogen atoms and optionally heteroatoms, such as S, O, N or P, alone or in combination.

The melting point of the at least one semi-crystalline polymer as disclosed herein can be less than 150° C. For example, the melting point of the at least one semi-crystalline polymer can range from 30° C. to 100° C. For instance, the melting point of the at least one semi-crystalline polymer can range from 30° C. to 60° C.

The at least one semi-crystalline polymer according to the present disclosure is solid at ambient temperature (25° C.) and atmospheric pressure (760 mmHg), the melting points is greater than or equal to 30° C. The melting point values correspond to the melting point measured using a differential scanning calorimeter (DSC), such as the calorimeter sold under the name DSC30 by Mettler, with a rise in temperature of 5° C. or 10° C. per minute (the melting point considered is the point corresponding to the temperature of the most endothermic peak of the thermogram).

The at least one semi-crystalline polymer according to the present disclosure can have a melting point which is greater than the temperature of the keratinous substrate intended to receive the composition, such as the skin or lips.

The at least one semi-crystalline polymer according to the present disclosure may be capable of structuring, alone or as a mixture, the composition without addition of a specific surfactant or of filler or of wax.

According to the present disclosure, the at least one semi-crystalline polymer can be, for example, soluble in the fatty phase, for instance in an amount of at least 1% by weight, at a temperature greater than their melting point. Apart from the at least one crystallizable chain or block, the blocks of the polymers are amorphous.

As used herein, the term “crystallizable chain or block” is understood to mean a chain or block which, if it were alone, would change reversibly from the amorphous state to the crystalline state, according to whether the temperature is above or below the melting point. A crystallizable chain within the meaning of the present disclosure is a group of atoms which is in the pendent or side position with respect to the backbone of the polymer. A cystallizable block is a group of atoms belonging to the backbone, a group constituting at least one of the repeat units of the polymer.

In one embodiment of the present disclosure, the polymer backbone of the at least one semi-crystalline polymer can be, for instance, soluble in the fatty phase.

For example, the at least one crystallizable block or chain of the at least one semi-crystalline polymer can be present in an amount greater than or equal to 30% by weight, relative to the total weight of each polymer, such as greater than or equal to 40% by weight. The at least one semi-crystalline polymer with at least one crystallizable side chain can be chosen from homo- and copolymers. The at least one semi-crystalline polymer of the present disclosure with at least one crystallizable block can be chosen from block and multiblock copolymers. They can be obtained by polymerization of a monomer with reactive (or ethylenic) double bonds or by polycondensation. When the at least one semi-crystalline polymer of the present disclosure is a polymer with at least one crystallizable side chain, the at least one crystallizable side chain can be, for example, chosen from those in the statistical or random form.

For instance, the at least one semi-crystalline polymer of the present disclosure can be synthetic in origin. According to one embodiment of the present disclosure, the at least one semi-crystalline polymer of the present disclosue does not comprise a polysaccharide backbone.

Among the semi-crystalline polymers that can be used according to the present disclosure, non-limiting mention may be made of:
block copolymers of polyolefins with controlled crystallization, the monomers of which are disclosed in European Patent No. EP-A-0 951 897,
polycondensates, such as of aliphatic or aromatic or aliphatic/aromatic polyester type,
homo- or copolymers carrying at least one crystallizable side chain and homo- or copolymers carrying, in the backbone, at least one crystallizable block, such as those disclosed in U.S. Pat. No. 5,156,911,
homo- or copolymers carrying at least one crystallizable side chain with, for instance, at least one fluorinated group, such as disclosed in International Patent Application No. WO-A-01/19333,
and mixtures thereof.

In one embodiment of the present disclosure, in the previously described homo- or copolymers, the at least one crystallizable side chain or block, can be hydrophobic.

A) Semi-crystalline Polymers with Crystallizable Side Chains

Among the semi-crystalline polymers with crystallizable side chain that may be used as disclosed herein, non-limiting mention may be made of, for example, those described in U.S. Pat. No. 5,156,911 and International Patent Application No. WO-A-01/19333. These include homopolymers or copolymers comprising from 50% to 100% by weight of units resulting from the polymerization of at least one monomer carrying at least one crystallizable hydrophobic side chain. These homo- or copolymers can have any nature provided that they exhibit the conditions indicated below, in addition to being soluble or dispersible in the fatty phase by heating above their melting point M.P. They can result:
from the polymerization, for instance radical polymerization, of at least one monomer with at least one double bond or ethylenic monomers reactive with respect to polymerization, namely with a vinyl, (meth)acrylic or allyl group,
from the polycondensation of at least one monomers carrying co-reactive groups (carboxylic or sulphonic acid, alcohol, amine or isocyanate groups), such as, for example, polyesters, polyurethanes, polyethers, polyureas or polyamides.
   In general, the crystallizable units (chains or blocks) of the at least one semi-crystalline polymer according to the present disclosure can originate from at least one monomer with at least one crystallizable block or chain used for the manufacture of semi-crystalline polymers. These polymers can be chosen from, for example, the homopolymers and copolymers resulting from the polymerization of at least one monomer with at least one crystallizable chain chosen from those of formula (X): 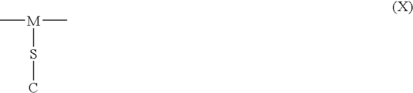
   wherein M is an atom of the polymer backbone,
S is a spacer, and
C is a crystallizable group

The crystallizable chains “—S—C” can be chosen from aliphatic and aromatic, and optionally fluorinated or perfluorinated. “S” can be chosen from, for example, linear, branched and cyclic (CH₂)ₙ, (CH₂CH₂O)ₙ, and (CH₂O) groups, wherein n is an integer ranging from 0 to 22. For instance, “S” can be a linear group. For further example, “S” and “C” may be different.

When the crystallizable chains are hydrocarbon aliphatic chains, they comprise hydrocarbon alkyl chains with at least 11 carbon atoms and at most 40 carbon atoms, such as 24 carbon atoms. They can be, for example, chosen from aliphatic chains and alkyl chains comprising at least 12 carbon atoms, for instance C₁₄-C₂₄ chains, such as C₁₆-C₂₂, alkyl chains. When they are fluorinated or perfluorinated alkyl chains, they comprise at least 11 carbon atoms, at least 6 carbon atoms of which are fluorinated.

Mention may be made, as non-limiting examples of semi-crystalline homopolymers or copolymers with crystallizable chain(s) that may be used as disclosed herein, of those resulting from polymerization of at least one monomer chosen from: saturated alkyl (meth)acrylates with a C₁₄-C₂₄ alkyl group, perfluoroalkyl (meth)acrylates with a C₁₁-C₁₅ perfluoroalkyl group, N-alkyl(meth)acrylamides with a C₁₄ to C₂₄ alkyl group, with or without a fluorine atom, vinyl esters with alkyl or perfluoro(alkyl) chains with a C₁₄ to C₂₄ alkyl group (with at least 6 fluorine atoms per one perfluoroalkyl chain), vinyl ethers with alkyl or perfluoro(alkyl) chains with a C₁₄ to C₂₄ alkyl group and at least 6 fluorine atoms per one perfluoroalkyl chain, C₁₄ to C₂₄ α-olefins, such as, for example, octadecene, para-alkylstyrenes with an alkyl group comprising from 12 to 24 carbon atoms, and mixtures thereof.

When the polymers result from a polycondensation, the crystallizable hydrocarbon and/or fluorinated chains as defined above are carried by a monomer which can be a diacid, a diol, a diamine or a diisocyanate.

When the polymers as disclosed herein are copolymers, they can additionally comprise from 0% to 50% of Y or Z groups resulting from the copolymerization:
α) of Y, which is chosen from polar and nonpolar monomers, and mixtures of the two:
When Y is a polar monomer, it is chosen from monomers carrying polyoxyalkylenated groups (for instance oxyethylenated and/or oxypropylenated groups), hydroxyalkyl (meth)acrylates, such as hydroxyethyl acrylate, (meth)acrylamide, N-alkyl(meth)acrylamides, N,N-dialkyl(meth)acrylamides, such as, for example, N,N-diisopropylacrylamide or N-vinylpyrrolidone (NVP), N-vinylcaprolactams, or monomers carrying at least one carboxylic acid group, such as (meth)acrylic acid, crotonic acid, itaconic acid, maleic acid or fumaric acid, or carrying a carboxylic acid anhydride group, such as maleic anhydride, and their mixtures.
When Y is a nonpolar monomer, it can be chosen from esters of the linear, branched and cyclic alkyl (meth)acrylate type, vinyl esters, alkyl vinyl ethers, α-olefins, styrenes, styrenes substituted by a C₁-C₁₀ alkyl group, such as α-methylstyrene, or a macromonomer of the polyorganosiloxane type with vinyl unsaturation.

As used herein, the term “alkyl” is understood to mean a saturated group, for instance a C₈-C₂₄ group, unless specifically mentioned.

β) of Z, which is a polar monomer or a mixture of polar monomers. In this case, Z has the same definition as the “polar Y” defined above.

For example, the semi-crystalline polymers with a crystallizable side chain can be chosen from alkyl (meth)acrylate and alkyl(meth)acrylamide homopolymers with an alkyl group as defined above, such as a C₁₄-C₂₄ alkyl group, copolymers of these monomers with a hydrophilic monomer, for instance different in nature from (meth)acrylic acid, such as N-vinylpyrrolidone or hydroxyethyl (meth)acrylate, and mixtures thereof.

For instance, the semi-crystalline polymer or polymers with a crystallizable side chain can have a weight-average molecular mass M_{w} ranging from 5,000 to 1,000,000, such as from 10,000 to 800,000, for example from 15,000 to 500,000, such as from 100,000 to 200,000.

Mention may be made, as non-limiting example of a semi-crystalline polymer which can be used in the composition according to the present disclosure, of the Intelimer® products from Landec described in the brochure “Intelimer® polymers”, Landec IP22 (Rev. 4-97). These polymers are in the solid form at ambient temperature (25° C.). They carry crystallizable side chains and exhibit the above formula (X).

For example, in one embodiment of the present disclosure, the semi-crystalline polymer is made of the Intelimer® product IPA 13-1 from Landec, which is a poly(stearyl acrylate) with a molecular weight of approximately 145,000 and a melting point of 49° C.

The semi-crystalline polymers can also be, by way further non-limiting example those disclosed in Example Nos. 3, 4, 5, 7 and 9 of U.S. Pat. No. 5,156,911 comprising a —COOH group, resulting from the copolymerization of acrylic acid and of C₅ to C₁₆ alkyl (meth)acrylate with a melting point ranging from 20° C. to 35° C., such as those resulting from the copolymerization:
of acrylic acid, of hexadecyl acrylate and of isodecyl acrylate in a 1/16/3 ratio,
of acrylic acid and of pentadecyl acrylate in a 1/19 ratio,
of acrylic acid, of hexadecyl acrylate and of ethyl acrylate in a 2.5/76.5/20 ratio,
of acrylic acid, of hexadecyl acrylate and of methyl acrylate in a 5/85/10 ratio,
of acrylic acid and of octadecyl methacrylate in a 2.5/97.5 ratio.

Further non-limiting mention can also be made of the polymer Structure “O” from National Starch, such as that disclosed in U.S. Pat. No. 5,736,125, with a melting point of 44° C.

The semi-crystalline polymers can be, for instance, semi-crystalline polymers with crystallizable pendent chains comprising fluorinated groups, such as those disclosed in Example Nos. 1, 4, 6, 7 and 8 of International Patent Application No. WO-A-01/19333.

Non-limiting mention may also be made of the semi-crystalline polymers obtained by copolymerization of stearyl acrylate and of acrylic acid or of NVP as disclosed in U.S. Pat. No. 5,519,063, or European Patent No. EP-A-0 550 745. In addition, non-limiting mention may also be made of the semi-crystalline polymers obtained by copolymerization of behenyl acrylate and of acrylic acid or of NVP as disclosed in U.S. Pat. No. 5,519,063 and European Patent No. EP-A-0 550 745.

B) Polymers Comprising, in the Backbone, at Least One Crystallizable Block

Polymers comprising, in the backbone, at least one crystallizable block are polymers which are soluble or dispersible in the fatty phase by heating above their melting point M.P. These polymers can be, for example, block copolymers comprised of at least two blocks of different chemical natures, one of which is crystallizable.

Among the polymers comprising, in the backbone, at least one crystallizable block that can be used as disclosed herein, non-limiting mention can be made of those chosen from block copolymers of olefin and of cycloolefin with a crystallizable chain, such as those resulting from the block polymerization of:
cyclobutene, cyclohexene, cyclooctene, norbornene (i.e., bicyclo[2.2.1]hept-2-ene), 5-methylnorbornene, 5-ethylnorbornene, 5,6-dimethylnorbornene,
5,5,6-trimethylnorbornene, 5-ethylidenenorbornene, 5-phenylnorbornene, 5-benzylnorbornene, 5-vinylnorbornene, 1,4,5,8-dimethano-1,2,3,4,4a,5,8a-octahydronaphthalene, dicyclopentadiene or mixtures thereof, with
ethylene, propylene, 1-butene, 3-methyl-1-butene, 1-hexene, 4-methyl-1-pentene, 1-octene, 1-decene, 1-eicosene or mixtures thereof,
   for instance copoly(ethylene/norbornene) blocks and (ethylene/propylene/ethylidenenorbornene) terpolymer blocks. Non-limiting mention may also be made of those resulting from the block copolymerization of at least two C₂-C₁₆ α-olefins, for instance C₂-C₁₂ α-olefins, such as those mentioned above, and for example, the block bipolymers of ethylene and 1-octene.

The polymer carrying, in the backbone, at least one crystallizable block can be chosen from copolymers exhibiting at least one crystallizable block, the remainder of the copolymer being amorphous at ambient temperature. These copolymers can, in addition, exhibit two crystallizable blocks of different chemical natures. The copolymers can be those which have, at ambient temperature, both a crystallizable block and a block which in both hydrophobic and lipophilic amorphous blocks are sequentially distributed. Non-limiting mention may be made, for example, of the polymers having one of the following crystallizable blocks and one of the following amorphous blocks:
Block crystallizable by nature of polyester type, such as poly(alkylene terephthalate)s, or of polyolefin type, such as polyethylenes or polypropylenes.
Amorphous and lipophilic block, such as amorphous polyolefins or copoly(olefin)s, for example poly(isobutylene); hydrogenated polybutadiene or hydrogenated poly(isoprene).

Mention may be made, as non-limiting examples of such copolymers with a crystallizable block and with an amorphous block, of:
a) Poly(ε-caprolactone)-b-poly(butadiene) block copolymers, for instance, which can be hydrogenated, such as those described in the paper, “Melting behavior of poly(β-caprolactone)-block-polybutadiene copolymers,” by S. Nojima, Macromolecules, 32, 3727-3734 (1999).
β) Block or multiblock hydrogenated poly(butylene terephthalate)-b-poly(isoprene) block copolymers, cited in the paper, “Study of morphological and mechanical properties of PP/PBT,” by B. Boutevin et al., Polymer Bulletin, 34, 117-123 (1995).
γ) The poly(ethylene)-b-copoly(ethylene/propylene) block copolymers cited in the papers, “Morphology of semi-crystalline block copolymers of ethylene-(ethylene-alt-propylene),” by P. Rangarajan et al., Macromolecules, 26, 4640-4645 (1993), and “Polymer aggregates with crystalline cores: the system poly(ethylene)-poly(ethylene-propylene),” P. Richter et al., Macromolecules, 30, 1053-1068 (1997).
δ) The poly(ethylene)-b-poly(ethylethylene) block copolymers cited in the general article, “Crystallization in block copolymers,” by I. W. Hamley, Advances in Polymer Science, vol. 148, 113-137 (1999).
   C) Polycondensates of Aliphatic or Aromatic or Aliphatic/Aromatic Polyester Type

The polyester polycondensates can be chosen from aliphatic polyesters. Their molecular mass can be, for example, ranging from 200 to 10,000, for instance ranging from 300 to 5,000, such as from 500 to 2,000 g/mol.

The polyester polycondensates can be chosen, for instance, from polycaprolactones. For example, the polycaprolactones can be chosen from ε-caprolactone homopolymers. Homopolymerization can be initiated with a diol, such as a diol comprising from 2 to 10 atoms, such as diethylene glycol, 1,4-butanediol or neopentyl glycol.

Non-limiting mention may be made, for example, among the polycaprolactones, of those sold under the names Capa® 240 (melting point of 68° C. and molecular weight of 4,000), Capa® 223 (melting point of 48° C. and molecular weight of 2,000), Capa® 222 (melting point of 48° C. and molecular weight of 2,000), Capa® 217 (melting point of 44° C. and molecular weight of 1,250), Capa® 2125 (melting point of 45° C. and molecular weight of 1,250), Capa® 212 (melting point of 45° C. and molecular weight of 1,000), Capa® 210 (melting point of 38° C. and molecular weight of 1,000) and Capa® 205 (melting point of 39° C. and molecular weight of 830) by Solvay and PCL-300 and PCL-700 by Union Carbide.

For example, in one embodiment of the present disclosure, Capa® 2125 is used, the melting point of which can range from 35° C. to 45° C., and the weight-average molecular mass of which is equal to 1250.

The semi-crystalline polymers of the composition of the present disclosure may or may not be partially crosslinked provided that the degree of crosslinking is not harmful to their dissolution or dispersion in the fatty phase by heating above their melting point. The crosslinking can then be chemical crosslinking, by reaction with a multifunctional monomer during the polymerization. It can also be physical crosslinking, which can then be due either to the establishment of bonds of hydrogen or dipolar type between groups carried by the polymer, such as, for example, dipolar interactions between carboxylate ionomers, these interactions being low in degree and carried by the backbone of the polymer, or to phase separation between the crystallizable blocks and the amorphous blocks carried by the polymer.

For example, in one embodiment of the present disclosure, the semi-crystalline polymers of the composition as disclosed herein are not crosslinked. The at least one semi-crystalline polymer can be present in the composition, in a total amount, ranging from 0.1% to 80% by weight, relative to the total weight of the composition, such as from 0.5% to 40%, for instance from 3% to 30% by weight. For example, it can be present in an amount ranging from 5% to 25% by weight, relative to the total weight of the composition.

Aqueous Phase

The composition according to the present disclosure can comprise at least one aqueous medium, constituting an aqueous phase, which can form the continuous phase of the composition.

The aqueous phase can be composed essentially of water. It can also comprise water and at least one water-miscible organic solvent (miscibility in water of greater than 50% by weight at 25° C.), such as lower monoalcohols comprising from 1 to 5 carbon atoms, for example ethanol or isopropanol, glycols comprising from 2 to 8 carbon atoms, such as propylene glycol, ethylene glycol, 1,3-butylene glycol or dipropylene glycol, C₃-C₄ ketones and C₂-C₄ aldehydes.

The aqueous phase (water and optionally at least one water-miscible organic solvent) can be present in an amount ranging from 1% to 95% by weight, for instance ranging from 3% to 80% by weight, such as ranging from 5% to 60% by weight, relative to the total weight of the composition.

The aqueous phase can, if desired, be thickened, gelled or structured by additionally incorporating therein a conventional aqueous gelling agent, for instance of inorganic origin, such as clay, for example, and/or of organic origin, such as an aqueous gelling polymer.

Such a medium can also comprise at least one volatile oil as defined below.

Fatty Phase

The composition, for example, when it is intended to be applied to the lips, can comprise at least one fatty phase, such as at least one fatty substance which is liquid at ambient temperature (25° C.) and at atmospheric pressure and/or one fatty substance which is solid at ambient temperature and at atmospheric pressure, such as waxes, gums and mixtures thereof. The fatty phase can additionally comprise gelling and structuring agents for oils of organic nature and/or organic solvents which are lipophilic.

According to one alternative embodiment of the present disclosure, the cosmetic composition does not comprise any paraffin, petrolatum or lanolin substances. Lanolins can exhibit the disadvantage of being sensitive to heat and to ultraviolet radiation and have a tendency to oxidize over time with the release of an unpleasant smell, which limits their use in cosmetic compositions. Furthermore, when lanolins are used in combination with oils commonly used in the cosmetics field, the compositions obtained can exhibit problems of tackiness, which can become more pronounced as the oil used increases in viscosity.

The fatty phase of the composition according to the present disclosure can, for example comprise, as liquid fatty substance, at least one oil chosen from volatile and nonvolatile oils.

As used herein, the term “volatile oil” is understood to mean any oil capable of evaporating on contact with the skin in less than one hour at ambient temperature and atmospheric pressure. The volatile oils of the present disclosure are volatile cosmetic oils which are liquid at ambient temperature and which have a nonzero vapor pressure, at ambient temperature and atmospheric pressure, ranging, for instance, from 0.01 mm Hg to 300 mm Hg (1.33 Pa to 40,000 Pa), such as greater than 0.3 mmHg (30 Pa).

As used herein, the term “nonvolatile oil” is understood to mean an oil which remains on the skin at ambient temperature and atmospheric pressure for at least several hours and which has, for instance, a vapor pressure of less than 0.01 mmHg (1.33 Pa).

These volatile or nonvolatile oils can be hydrocarbon oils, such as of animal or vegetable origin, silicone oils, and mixtures thereof. As used herein, the term “hydrocarbon oil” is understood to mean an oil mainly comprising hydrogen and carbon atoms and optionally oxygen, nitrogen, sulphur and/or phosphorus atoms. The volatile hydrocarbon oils can be chosen from hydrocarbon oils comprising from 8 to 16 carbon atoms, for instance branched C₈-C₁₆ alkanes, such as C₈-C₁₆ isoalkanes of petroleum origin (also known as isoparaffins), for example isododecane (also known as 2,2,4,4,6-pentamethylheptane), isodecane, isohexadecane and, for example, the oils sold under the trade names of Isopars® or Permetyls®, branched C₈-C₁₆ esters, such as isohexyl neopentanoate, and mixtures thereof. Other volatile hydrocarbon oils, such as oil distillates, for instance those sold under the name Shell Solt® by Shell, can also be used.

Non-limiting mention may also be made, as volatile oils, of volatile silicones, such as, for example, volatile linear or cyclic silicone oils, for instance those having a viscosity≦8 centistokes (8×10⁻⁶ m²/s), comprising for example from 2 to 7 silicon atoms, these silicones optionally comprising alkyl or alkoxy groups comprising from 1 to 10 carbon atoms. Non-limiting mention may further be made, for example, among the volatile silicone oils which can be used as disclosed herein, of octamethylcyclotetrasiloxane, decamethylcyclopentasiloxane, dodecamethylcyclohexasiloxane, heptamethylhexyltrisiloxane, heptamethyloctyltrisiloxane, hexamethyldisiloxane, octamethyltrisiloxane, decamethyltetrasiloxane, dodecamethylpentasiloxane and mixtures thereof.

The volatile oil can be present in the composition according to the present disclosure in an amount ranging from 0.1% to 98% by weight, for instance, from 1% to 65% by weight, such as from 2% to 50% by weight, relative to the total weight of the composition.

The nonvolatile oils can be chosen from, for example, nonvolatile hydrocarbon oils, optionally fluorinated, and/or nonvolatile silicone oils.

Mention may be made, as non-limiting examples of nonvolatile hydrocarbon oil, of:
hydrocarbon oils of animal origin,
hydrocarbon oils of vegetable origin, such as triglycerides composed of esters of fatty acids and of glycerol, the fatty acids of which can have various chain lengths ranging from C₄ to C₂₄, it being possible for these chains to be linear or branched and saturated or unsaturated; these oils include, for example, wheat germ, sunflower, grape seed, sesame, maize, apricot, castor, karite, avocado, olive, soybean, sweet almond, palm, rapeseed, cottonseed, hazelnut, macadamia, jojoba, alfalfa, poppy, pumpkinseed, sesame, cucumber, blackcurrant seed, evening primrose, millet, barley, quinoa, rye, safflower, candlenut, passionflower or musk rose oil; karite butter; or triglycerides of caprylic/capric acids, such as those sold by the company Stearineries Dubois or those sold under the names Miglyol 810®, 812®, and 818® by Dynamit Nobel,
synthetic ethers comprising from 10 to 40 carbon atoms,
linear or branched hydrocarbons of mineral or synthetic origin, such as liquid petrolatum, polydecenes, hydrogenated polyisobutene, such as parleam, squalane, and their mixtures,
synthetic esters, such as oils of formula R₁COOR₂ wherein R₁ is chosen from residues of linear and branched fatty acids comprising from 1 to 40 carbon atoms and R₂ is chosen from hydrocarbon chains, for example a branched hydrocarbon chain, comprising from 1 to 40 carbon atoms, provided that R₁+R₂ is ≧10, such as, for example, Purcellin oil (cetostearyl octanoate), isopropyl myristate, isopropyl palmitate, C₁₂ to C₁₅ alkyl benzoates, hexyl laurate, diisopropyl adipate, isononyl isononanoate, 2-ethylhexyl palmitate, isostearyl isostearate, or heptanoates, octanoates, decanoates or ricinoleates of alcohols or of polyalcohols, such as propylene glycol dioctanoate; hydroxylated esters, such as isostearyl lactate or diisostearyl malate; esters of polyols and esters of pentaerythritol,
fatty alcohols which are liquid at ambient temperature with a branched and/or unsaturated carbon chain comprising from 12 to 26 carbon atoms, such as octyldodecanol, isostearyl alcohol, oleyl alcohol, 2-hexyldecanol, 2-butyloctanol and 2-undecylpentadecanol,
higher fatty acids, such as oleic acid, linoleic acid or linolenic acid, and mixtures thereof.

The nonvolatile silicone oils which can be used in the composition according to the present disclosure can also be nonvolatile polydimethylsiloxanes (PDMSs), polydimethylsiloxanes comprising pendant alkyl or alkoxy groups and/or alkyl or alkoxy groups at the ends of the silicone chain, which groups each comprise from 2 to 24 carbon atoms, or phenylated silicones, such as phenyl trimethicones, phenyl dimethicones, phenyl(trimethylsiloxy)diphenylsiloxanes, diphenyl dimethicones, diphenyl(methyldiphenyl)trisiloxanes and (2-phenylethyl)trimethylsiloxysilicates, and mixtures thereof.

The nonvolatile oils can be present in the composition according to the present disclosure in an amount ranging from 0.01% to 90% by weight, for instance from 0.1% to 85% by weight, such as from 1% to 70% by weight, relative to the total weight of the composition.

The oils can be present in an amount ranging from 0.01% to 99%, relative to the total weight of the composition, for instance from 0.05% to 60%, such as from 1% to 35%.

The fatty substance which is solid at ambient temperature and at atmospheric pressure, can be chosen from waxes, gums and mixtures thereof. This solid fatty substance can be present in an amount ranging from 0.01% to 50%, for instance, from 0.1% to 40%, such as from 0.2% to 30% by weight, relative to the total weight of the fatty phase.

The waxes can be chosen from hydrocarbon, fluorinated and/or silicone waxes and can be of vegetable, mineral, animal and/or synthetic origin. For instance, the waxes can exhibit a melting point of greater than 45° C.

Non-limiting mention may be made, among the waxes that can be used in the composition of the present disclosure, of beeswax, carnauba wax, candelilla wax, paraffin wax, microcrystalline waxes, ceresin or ozokerite; synthetic waxes, such as polyethylene or Fischer-Tropsch waxes; or silicone waxes, such as alkyl or alkoxy dimethicones comprising from 16 to 45 carbon atoms.

The total amount of all wax that can be present in the composition can range, for example, from 15% to 35%, for instance from 20% to 30% by weight, relative to the total weight of the composition.

The composition according to the present disclosure can also comprise at least one pasty compound.

The composition can also comprise at least one gum. Such gums are generally provided in a form dissolved in an oil.

As used herein, the term “gum” is understood to mean a fatty substance which exists in the form of a polymer which has a weight-average molecular weight ranging from 50,000 to 1,000,000. The gum is often sold in dispersion in an organic solvent of the silicone oil type.

The nature and the amounts of the gums or waxes depend on the mechanical properties and textures desired. By way of indication, for example, the at least one gum can be present in an amount ranging from 0.01% to 50%, for instance, from 2% to 40%, such as from 5% to 30% by weight, relative to the total weight of the composition.

Fillers and Pigments

The compositions as disclosed herein can further comprise at least one filler. As used herein, the term “fillers” is understood to mean any organic and/or inorganic compound introduced into the cosmetic composition in order to adjust its properties in terms of texture, or in other words to control its rheological properties. Pigments and pearlescent agents, for instance, are excluded from this definition.

According to one alternative embodiment of the present disclosure, the at least one filler can be present in the cosmetic composition in an amount less than or equal to 15% by weight, for instance, less than or equal to 10% by weight, such as less than or equal to 7% by weight, relative to the total weight of the composition.

The at least one filler can be chosen one, for example, spherical filler(s), such as, for example, talc, zinc stearate, mica, kaolin, polyamide (Nylon®) powders (Orgasol® from Atochem), polyethylene powders, tetrafluoroethylene polymer (Teflon®) powders, starch, boron nitride, polymer microspheres, such as those of poly(vinylidene chloride)/acrylonitrile, for example Expancel® (Nobel Industrie), or of acrylic acid copolymers (Polytrap® from Dow Corning), silicone resin microbeads (Tospearls® from Toshiba, for example) and organopolysiloxane elastomers.

The composition according to the present disclosure can furthermore comprise at least one emulsifying surface-active agent, which can be present in an amount ranging from 0.1% to 30% by weight, such as ranging from 5% to 15% by weight, relative to the total weight of the composition.

The at least one surface-active agent can be chosen from anionic and nonionic surface-active agents. Reference may be made to the document “Encyclopedia of Chemical Technology, Kirk-Othmer,” volume 22, p. 333-432, 3rd edition, 1979, Wiley, for the definition of the properties and functions (emulsifying) of surfactants, for instance, p. 347-377 of this reference for anionic and nonionic surfactants.

Among the surfactants that may be used in the composition according to the present disclosure, non-limiting mention may be made of:
nonionic surfactants: fatty acids, fatty alcohols, polyethoxylated or polyglycerolated fatty alcohols, such as polyethoxylated stearyl or cetearyl alcohols, esters of a fatty acid and of sucrose, alkyl glucose esters, for instance polyoxyethylenated fatty esters of C₁-C₆ alkyl glucose, and mixtures thereof;
anionic surfactants: C₁₆-C₃₀ fatty acids neutralized with amines, aqueous ammonia or alkaline salts, and mixtures thereof.

For example, use can be made of surfactants which make it possible to obtain an oil-in-water or wax-in-water emulsion.

The compositions of the present disclosure can also comprise at least one coloring agent. The at least one coloring agent can be, for example, present in an amount ranging from 0.01% to 40% by weight, for instance, from 0.01% to 30% by weight, such as from 0.05% to 25% by weight, relative to the total weight of the composition.

The at least one coloring agent can be chosen from pigments, water-soluble and fat-soluble dyes, and pearlescent agents.

As used herein, the term “pigments” is understood to mean white or colored and inorganic or organic particles which are insoluble in the liquid hydrophilic phase and which are intended to color and/or opacify the composition. In addition, the term “fillers” is also understood to mean colorless or white, inorganic or synthetic and lamellar or nonlamellar particles. As used herein, the term “pearlescent agents” is understood to mean iridescent particles, for instance produced by certain shellfish in their shells, or else synthesized.

The at least one pigment can be present in the composition in an amount ranging from 0.01% to 25% by weight, for instance, from 0.01% to 15% by weight, such as from 0.02% to 5% by weight, relative to the total weight of the composition.

Non-limiting mention may be made, among the inorganic pigments which can be used in the present disclosure, of titanium, zirconium or cerium oxides and zinc, iron or chromium oxides, ferric blue, manganese violet, ultramarine blue and chromium hydrate. Non-limiting mention may be made, among organic pigments which can be used in the present disclosure, of carbon black, pigments of D & C type, lakes based on cochineal carmine of barium, strontium, calcium or aluminium, or the diketopyrrolopyrroles (DPP) disclosed in European Patent Nos. EP-A-542 669, EP-A-787 730, and EP-A-787 731, and International Patent Application No. WO-A-96/085737.

The at least one pearlescent agent can be present in the composition in an amount ranging from 0.01% to 25% by weight, for instance, from 0.01% to 15% by weight, such as from 0.02% to 5% by weight, relative to the total weight of the composition.

The at least one pearlescent pigment can be chosen from white pearlescent pigments, such as mica covered with titanium oxide or with bismuth oxychloride, colored pearlescent pigments, such as titanium oxide-coated mica with iron oxides, titanium oxide-coated mica with, for example, ferric blue or chromium oxide, or titanium oxide-coated mica with an organic pigment of the abovementioned type, and pearlescent pigments based on bismuth oxychloride.

The composition can also comprise at least one water-soluble or fat-soluble dye, which can be present in an amount ranging from 0.01% to 6% by weight, relative to the total weight of the composition, such as ranging from 0.01% to 3% by weight. The fat-soluble dyes include, for example, Sudan red, DC Red 17, DC Green 6, p-carotene, soybean oil, Sudan brown, DC Yellow 11, DC Violet 2, DC Orange 5 and quinoline yellow. The water-soluble dyes include, for example, beetroot juice and methylene blue.

Additives

The composition according to the present disclosure can additionally comprise at least one additive conventionally used in the fields under consideration, such as in the cosmetics and dermatological fields. These ingredients can be chosen from, for example, vitamins, antioxidants, thickeners, trace elements, softening agents, sequestering agents, fragrances, basifying or acidifying agents, preservatives, UV screening agents, hydrophilic or lipophilic active principles, and mixtures thereof. The at least one additive can be present in an amount conventionally used in the fields under consideration, for example in an amount ranging from 0.01% to 20% by weight, relative to the total weight of the composition.

Of course, a person skilled in the art will take care to chose any of the optional additional compounds and/or their amounts so that the beneficial properties of the composition according to the present disclosure are not, or not substantially, detrimentally affected by the addition under consideration.

The composition of the present disclosure can be obtained according to preparation processes conventionally used in cosmetics or in dermatology.

The composition of the of the present disclosure can be provided in the form of a solid, pasty or liquid composition, the solid composition being compacted or cast as a stick or in a dish. For example, it can be provided in the solid form, namely in the hard form (which does not flow under its own weight), such as cast or compacted, for example as a stick or in a dish. In this case, it can be provided in the form of lipsticks, lip balms, cast foundations, concealers, complexion “correctors” or “embellishers”, eye shadows and/or face powders.

However, it can also be provided in the form of a paste, solid or cream. It can be an oil-in-water or water-in-oil emulsion, a solid or soft anhydrous gel or can be in the form of a free or compact powder and can even be in the two-phase form. According to one embodiment of the present disclosure, it is provided in the form of an emulsion.

The composition according to the present disclosure can be provided in the form of a colored or colorless composition, in the form of a sun protection or make-up removal composition or in the form of a hygiene composition. The compositions can further comprise at least one cosmetic active ingredient. If it comprises, for instance, cosmetic active ingredients, it can then be used as care or treatment base for the skin, such as the hands or the face, or for the lips (lip balms, which protect the lips from cold and/or the sun and/or the wind) or as deodorant. Non-limiting mention may be made, among the cosmetic active ingredients that can be used as disclosed herein, of vitamins A, E, C and B3, provitamins, such as D-panthenol, soothing active principles, such as α-bisabolol, aloe vera, allantoin, plant extracts or essential oils, protecting or restructuring agents, such as ceramides, freshness active principles, such as menthol and its derivatives, emollients (cocoa butter, dimethicone), moisturizing agents (arginine PCA), anti-wrinkle active principles, essential fatty acids, and mixtures thereof.

The composition of the present disclosure can also be provided in the form of a product for making up the skin, for instance of the face, such as a foundation, a blusher or a paint, such as a semi-permanent tattooing product, or for making up the lips, such as a lipstick or a lip gloss, optionally exhibiting care or treatment properties, or a product for making up the superficial body growths, such as, for example, a nail varnish, a mascara, an eyeliner or a product for coloring or caring for the hair.

Of course, the composition of the invention must be cosmetically acceptable, that is nontoxic and capable of being applied to the skin, superficial body growths or lips of human beings.

Other than in the operating examples, or where otherwise indicated, all numbers expressing quantities of ingredients, reaction conditions, and so forth used in the specification and claims are to be understood as being modified in all instances by the term “about.” Accordingly, unless indicated to the contrary, the numerical parameters set forth in the following specification and attached claims are approximations that may vary depending upon the desired properties sought to be obtained by the present disclosure. At the very least, and not as an attempt to limit the application of the doctrine of equivalents to the scope of the claims, each numerical parameter should be construed in light of the number of significant digits and ordinary rounding approaches.

Notwithstanding that the numerical ranges and parameters setting forth the broad scope of the disclosure are approximations, the numerical values set forth in the specific examples are reported as precisely as possible. Any numerical value, however, inherently contain certain errors necessarily resulting from the standard deviation found in their respective testing measurements.

The following examples are intended to illustrate the present disclosure in a non-limiting manner.

### EXAMPLES

### Example 1

### Lipstick

| Component | Percentage by weight |
|---|---|
| Polymer dispersion* | 30 |
| Triglyceride of 2-decyltetradecanoic | 2.02 |
| acid | |
| Dimer dilinoleyl diol/dimer dilinoleic | 10 |
| copolymers (Lusplan DD-DA5) | |
| Octyldodecanol | 9 |
| Preservatives | 0.47 |
| Polycaprolactone with an MW of 1250 | 9 |
| g/mol (Capa 1215 from Solvay) | |
| Poly(vinyl laurate) | — |
| Vinylpyrrolidone/eicosene copolymer | 6 |
| Microcrystalline wax | 10 |
| Polyethylene wax | 2 |
| Polymethylene wax, M.p. 40° C. | 10 |
| Pigments | 6.03 |
| Dimethicone-coated silica | 5 |
| Fragrance | 0.48 |
| Total | 100 |

*Synthesis of the dispersion of polymer particles
A dispersion of noncrosslinked copolymer of methyl acrylate and of acrylic acid in a ratio of 85/15 in heptane was prepared according to the method of Example 1 of European Patent No. EP-A-749 746. When the polymerization was complete, hydrogenated polyisobutene was added and the heptane was distilled off under vacuum.
A dispersion of poly(methyl acrylate/acrylic acid) particles stabilized in hydrogenated polyisobutene by a polystyrene/copoly(ethylene-propylene) sequential diblock copolymer, sold under the name of Kraton G1701, was thus obtained with a solids content of 21% by weight and a mean particle size of 150 nm.

Procedure for the Preparation of the Lipstick

All the starting materials were weighed into a jacketed heating vessel with circulation of oil and were then heated with stirring (turbine mixer).

After the materials were completely melted and the mixture was homogenized, the latter was milled 5 times in succession in a triple roll mill. The paste obtained was stabilized at 20° C. for 24 hours and was then packaged in small pots.

Evaluation (in vitro):

The formulation 1 was tested in vitro according to the test described above, which consists in evaluating the resistance of the formulation to water and to oil. The results were as follows:

| | Example 1 |
|---|---|
| | (polycaprolactone) |
| Resistance to pressure | 91.48 |
| Resistance to pressure + wiping | 53.96 |

### Example 2

### Lipstick

| Component | Percentage by weight |
|---|---|
| Triisocetyl citrate | 9.78 |
| Octyldodecanol | 15.59 |
| Stearate of poly(12-hydroxystearic acid), | 1.90 |
| Solsperse 21 000 from Avecia | |
| Isononyl isononanoate | 11.12 |
| Isopropyl isostearate | 19.00 |
| Squalane | 3.00 |
| Dimer dilinoleyl diol/dimer dilinoleic copolymers | 6.00 |
| (Lusplan DD-DA5) | |
| Pigments | 7.3 |
| Kaolin | 3.30 |
| Poly(methyl methacrylate) | 0.50 |
| Nylon | 2 |
| Hydrogenated cocoglycerides | 2 |
| Poly(stearyl acrylate) (Intelimer ® IPA 13-1 from | 7.50 |
| Landec) | |
| Poly(behenyl acrylate)* | 11 |
| *Preparation of poly(behenyl acrylate) | |
| 120 g of hydrogenated polyisobutene were introduced into a 1 l reactor equipped with a central anchor stirrer, a reflux condenser and a thermometer. The hydrogenated polyisobutene was heated from ambient temperature to 80° C. over 45 min. At 80° C., the following mixture C₁: 40 g of | |
| # cyclohexane + 4 g of Trigonox 141 [2,5-bis(2-ethylhexanoylperoxy)-2,5-dimethylhexane], was introduced over 2 hours. 30 min after starting to run the mixture C₁, the mixture C₂, composed of: 200 g of behenyl acrylate + 400 g of cyclohexane, was introduced over 1 hour and 30 minutes. | |

After the two mixtures were finished running, the reaction was allowed to take place for an additional 3 hours at 80° C. and then all the cyclohexane present in the reaction medium was distilled off at atmospheric pressure.
The polymer obtained was at 60% by weight, relative to the active material in Parleam oil. Its weight-average molecular mass ranged from 17,000 to 27,000 and its M.P. was 58° C.

1. A cosmetic composition comprising at least one ester of dimer diol and of at least one acid chosen from dicarboxylic acid and C₄ to C₃₄ monocarboxylic acid, and at least one semi-crystalline polymer with an organic structure, wherein the at least one semi-crystalline polymer has a melting point greater than or equal to 30° C.

2. The cosmetic composition according to claim 1, wherein the at least one semi-crystalline polymer is chosen from homopolymers and copolymers comprising units resulting from the polymerization of at least one monomer comprising at least one crystallizable hydrophobic side chain.

3. The cosmetic composition according to claim 1, wherein the at least one semi-crystalline polymer is chosen from the homopolymers and copolymers resulting from the polymerization of at least one monomer with at least one crystallizable chain of formula (X): 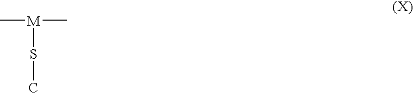
wherein M is an atom of the polymer backbone
S is a spacer, and
C is a crystallizable group,
and mixtures thereof, wherein “S—C” is chosen from optionally fluorinated and perfluorinated alkyl chains comprising at least 11 carbon atoms.

4. The cosmetic composition according to claim 1, wherein the at least one semi-crystalline polymer is chosen from the polymers resulting from the polymers resulting from the polymerization of at least one monomer chosen from acrylic acid, methacrylic acid, crotonic acid, itaconic acid, maleic acid, and maleic anhydride.

5. The cosmetic composition according to claim 1, wherein the at least one semi-crystalline polymer is chosen from the homopolymers and copolymers resulting from the polymerization of at least one monomer with a crystallizable chain chosen from saturated C₁₄-C₂₄ alkyl (meth)acrylates, C₁₁-C₁₅ perfluoroalkyl (meth)acrylates, N—(C₁₄ to C₂₄ alkyl)(meth)acrylamides, with or without a fluorine atom, vinyl esters comprising C₁₄ to C₂₄ alkyl and perfluoroalkyl chains, vinyl ethers comprising C₁₄ to C₂₄ alkyl and perfluoroalkyl chains, C₁₄ to C₂₄ α-olefins, and para-alkylstyrenes with an alkyl group comprising from 12 to 24 carbon atoms.

6. The cosmetic composition according to claim 5, wherein the at least one semi-crystalline polymer is chosen from homopolymers of alkyl (meth)acrylate and of alkyl(meth)acrylamide such that the alkyl group is a C₁₄ to C₂₄ alkyl group.

7. The cosmetic composition according to claim 6, wherein the at least one semi-crystalline polymer is chosen from homopolymers of alkyl (meth)acrylate such that the alkyl group is a C₁₆ to C₂₀ alkyl group.

8. The cosmetic composition according to claim 1, wherein the at least one semi-crystalline polymer has a weight-average molecular mass ranging from 5,000 to 1,000,000.

9. The cosmetic composition according to claim 8, wherein the at least one semi-crystalline polymer has a weight-average molecular mass ranging from 15,000 to 500,000.

10. The cosmetic composition according to claim 1, wherein the at least one semi-crystalline polymer is a polyester polycondensate.

11. The cosmetic composition according to claim 10, wherein the polyester polycondensate is chosen from polycaprolactones.

12. The cosmetic composition according to claim 10, wherein the polyester polycondensate has a molecular mass ranging from 200 g/mol to 10,000 g/mol.

13. The cosmetic composition according to claim 12, wherein the polyester polycondensate has a molecular mass ranging from 500 g/mol to 2,000 g/mol.

14. The cosmetic composition according to claim 1, wherein the at least one semi-crystalline polymer has a melting point ranging from 30° C. to 100° C.

15. The cosmetic composition according to claim 14, wherein the at least one semi-crystalline polymer has a melting point ranging from 30° C. to 60° C.

16. The cosmetic composition according to claim 1, wherein the at least one semi-crystalline polymer is present in an amount ranging from 0.1% to 80% by weight, relative to the total weight of the composition.

17. The cosmetic composition according to claim 16, wherein the at least one semi-crystalline polymer is present in an amount ranging from 5% to 25% by weight, relative to the total weight of the composition.

18. The cosmetic composition according to claim 1, wherein the monocarboxylic acids are chosen from:
saturated linear acids,
fatty acids,
hydroxy acids,
cyclic acids,
and mixtures thereof.

19. The cosmetic composition according to claim 18, wherein the saturated linear acids are chosen from butanoic acid, pentanoic acid, hexanoic acid, heptanoic acid, octanoic acid, nonanoic acid, decanoic acid, undecanoic acid, dodecanoic acid, tridecanoic acid, tetradecanoic acid, heptadecanoic acid, hexadecanoic acid, pentadecanoic acid, octadecanoic acid, nonadecanoic acid, eicosanoic acid, docosanoic acid, tetracosanoic acid, and mixtures thereof.

20. The cosmetic composition according to claim 18, wherein the hydroxy acids are chosen from 2-hydroxybutanoic acid, 2-hydroxypentanoic acid, 2-hydroxyhexanoic acid, 2-hydroxyheptanoic acid, 2-hydroxyoctanoic acid, 2-hydroxynonanoic acid, 2-hydroxydecanoic acid, 2-hydroxyundecanoic acid, 2-hydroxydodecanoic acid, 2-hydroxytridecanoic acid, 2-hydroxytetradecanoic acid, 2-hydroxyhexadecanoic acid, 2-hydroxyheptadecanoic acid, 2-hydroxyoctadecanoic acid, 12-hydroxyoctadecanoic acid, 2-hydroxynonadecanoic acid, 2-hydroxyeicosanoic acid, 2-hydroxydocosanoic acid, 2-hydroxytetracosanoic acid, and mixtures thereof.

21. The cosmetic composition according to claim 18, wherein the cyclic acids are chosen from cyclohexanoic acid, hydrogenated rosin, rosin, abietic acid, hydrogenated abietic acid, benzoic acid, p-hydroxybenzoic acid, p-aminobenzoic acid, cinnamic acid, p-methoxycinnamic acid, salicylic acid, gallic acid, pyrrolidonecarboxylic acid, nicotinic acid, and mixtures thereof.

22. The cosmetic composition according to claim 18, wherein the fatty acids are chosen from
branched fatty acids,
unsaturated linear C₈ to C₃₄ fatty acids,
fatty acids of natural origin,
and mixtures thereof.

23. The cosmetic composition according to claim 22, wherein the branched fatty acids are chosen from isobutanoic acid, isopentanoic acid, pivalic acid, isohexanoic acid, isoheptanoic acid, isooctanoic acid, dimethyloctanoic acid, isononanoic acid, isodecanoic acid, isoundecanoic acid, isododecanoic acid, isotridecanoic acid, isotetradecanoic acid, isopentadecanoic acid, isohexadecanoic acid, isoheptadecanoic acid, isooctadecanoic acid, isononadecanoic acid, isoeicosanoic acid, 2-ethylhexanoic acid, 2-butyloctanoic acid, 2-hexyldecanoic acid, 2-octyidodecanoic acid, 2-decyltetradecanoic acid, 2-dodecylhexadecanoic acid, 2-tetradecyloctadecanoic acid, 2-hexadecyloctadecanoic acid, and mixtures thereof.

24. The cosmetic composition according to claim 22, wherein the unsaturated linear C₈ to C₃₄ fatty acids are chosen from undecenoic acid, linderic acid, myristoleic acid, palmitoleic acid, oleic acid, linoleic acid, elaidinic acid, gadolenoic acid, eicoapentaenoic acid, docosahexaenoic acid, erucic acid, brassidic acid, arachidonic acid, and mixtures thereof.

25. The cosmetic composition according to claim 22, wherein the fatty acids of natural origin are chosen from fatty acids of orange oil, of avocado oil, of macadamia oil, of olive oil, of hydrogenated soybean oil, of jojoba oil, of palm oil, of castor oil, of wheat germ oil, of saffron oil, of cottonseed oil, of mink oil, and mixtures thereof.

26. The cosmetic composition according to claim 1, wherein the dicarboxylic acids are chosen from
compounds of formula (I) HOOC—(CH₂)ₙ—COOH wherein n is an integer ranging from 1 to 16, and
the dimer diacids obtained by dimerization of at least one unsaturated monocarboxylic acid.

27. The cosmetic composition according to claim 26, wherein the unsaturated monocarboxylic acid is an unsaturated C₈ to C₃₄ fatty acid.

28. The cosmetic composition according to claim 27, wherein the unsaturated C₈ to C₃₄ fatty acid is chosen from undecenoic acid, linderic acid, myristoleic acid, palmitoleic acid, oleic acid, linoleic acid, elaidinic acid, gadolenoic acid, eicosapentaenoic acid, docosahexaenoic acid, erucic acid, brassidic acid, arachidonic acid and mixtures thereof.

29. The cosmetic composition according to claim 26, wherein the dicarboxylic acid is a dimer diacid.

30. The cosmetic composition according to claim 29, wherein the dimer diacid is dilinoleic diacid.

31. The cosmetic composition according to claim 29, wherein the dimer diacid is saturated.

32. The cosmetic composition according to claim 1, wherein the dimer diol derives from the hydrogenation of a dimer diacid.

33. The cosmetic composition according to claim 32, wherein the dimer diacid derives from the dimerization of an unsaturated fatty acid.

34. The cosmetic composition according to claim 33, wherein the unsaturated fatty acid is chosen from undecenoic acid, linderic acid, myristoleic acid, palmitoleic acid, oleic acid, linoleic acid, elaidinic acid, gadolenoic acid, eicoapentaenoic acid, docosahexaenoic acid, erucic acid, brassidic acid and arachidonic acid.

35. The cosmetic composition according to claim 1, wherein the dimer diol derives from the hydrogenation of dilinoleic diacid.

36. The cosmetic composition according to claim 1, wherein the dimer diol is saturated.

37. The cosmetic composition according to claim 29, wherein the dimer diacid is identical to the dimer diacid from which the dimer diol derives.

38. The cosmetic composition according to claim 1, wherein the at least one ester is chosen from those of formula (II):
HO—R¹—(—OCO—R²—COO—R¹—)ₕ—OH (II)
wherein:
R¹ is chosen from dimer diol residues obtained by hydrogenation of dilinoleic diacid,
R² is chosen from hydrogenated dilinoleic diacid residues, and
h is an integer ranging from 1 to 9.

39. The cosmetic composition according to claim 1, wherein the at least one ester is present in an amount ranging from 1% to 99% by weight, relative to the total weight of the composition.

40. The cosmetic composition according to claim 39, wherein the at least one ester is present in an amount ranging from 10% to 35% by weight, relative to the total weight of the composition.

41. The cosmetic composition according to claim 1, wherein the composition is provided in the form of a product for caring for and/or making up the skin and/or lips.

42. The cosmetic composition according to claim 35, wherein the composition is provided in the form of a foundation, face powder, eye shadow, lipstick, base or balm for caring for the lips, concealer, eyeliner or mascara.

43. A method for making up and/or caring for the skin, lips and/or superficial body growths, comprising applying to the skin, lips and/or superficial body growths at least one composition comprising at least one ester of dimer diol and of at least one acid chosen from dicarboxylic acid and C₄ to C₃₄ monocarboxylic acid, and at least one semi-crystalline polymer with an organic structure, wherein the at least one semi-crystalline polymer has a melting point greater than or equal to 30° C.
